(19) United States
(12) Patent Application Publication
(10) Pub. No.: US 2014/0356308 A1
(43) Pub. Date: Dec. 4, 2014

(54) NON-AQUEOUS HAIR OIL COMPOSITION COMPRISING ISOPARAFFIN BASE OIL, SILICONE ELASTOMER AND ITS SOLUBILIZER

(71) Applicant: The Procter & Gamble Company, Cincinnati, OH (US)

(72) Inventors: Jun HASEGAWA, Singapore (JP); Taisuke OKU, Singapore (JP)

(73) Assignee: The Procter & Gamble Company, Cincinnati, OH (US)

(21) Appl. No.: 14/291,401

(22) Filed: May 30, 2014

Related U.S. Application Data
(60) Provisional application No. 61/828,691, filed on May 30, 2013.

(51) Int. Cl. A61K 8/898; A61Q 5/12

(52) U.S. Cl. CPC A61K 8/898; A61Q 5/12; A61K 2800/40
USPC 424/70.122

(57) ABSTRACT

Disclosed is a non-aqueous hair care composition comprising: a base oil being a volatile isoparaffin; a silicone elastomer; a solubilizer for the silicone elastomer which is a non-volatile silicone having a viscosity from about 25 to about 100,000 mm²·s⁻¹. The composition of the present invention has improved stability such as reduced phase separation at lower temperature, and also has a balanced dry performance.

### FIELD OF THE INVENTION

The present invention relates to a non-aqueous hair care composition comprising: a base oil being a volatile isoparaffin; a silicone elastomer; a solubilizer for the silicone elastomer which is a non-volatile silicone having a viscosity from about 25 to about 100,000 mm²·s⁻¹. The composition of the present invention has improved stability such as reduced phase separation at lower temperature, and also has a balanced dry performance.

### BACKGROUND OF THE INVENTION

A variety of approaches have been developed to condition the hair. A common method of providing conditioning benefits to the hair is through the use of application of hair conditioning composition. Conditioning formulations can be in the form of rinse-off products or leave-on products, and can be in the form of an emulsion, cream, gel, spray, mousse, oil, liquid and serum.

One of the leave-on products is a hair oil product which comprises a larger amount of oily compounds and is often non-aqueous liquid product. For example, Mirubon's JPA 2006-249002 relates to a non-aqueous hair treatment containing dimethiconol, aminosilicone, cyclic silicone and liquid paraffin. Mirubon's JPA 2006-249002 discloses that it is preferred that the amount of the dimethiconol is from 1.5 to 4.5 mass %, the amount of the aminosilicone is from 0.4 to 2 mass %, the amount of the cyclic silicone is from 80.5 to 98.09 mass %, and the amount of the liquid paraffin is 0.01 to 3mass %. Mirubon's JPA 2006-249002 also discloses exemplified compositions which further contains 0.05% of crosslinked methypolysiloxane with a tradename of KSG-15.

However, there is still a need for non-aqueous liquid hair oil product to provide balanced dry performance, i.e., providing dry conditioning such as smoothness, shine, and balanced hair volume while not providing greasiness/stickiness.

There is also a need for such non-aqueous oil compositions, especially when containing silicone elastomers such as crosslinked methypolysiloxane with a tradename of KSG-15, to have an improved stability such as reduced phase separation at lower temperature.

None of the existing art provides all of the advantages and benefits of the present invention.

### SUMMARY OF THE INVENTION

The present invention is directed to a non-aqueous hair care composition comprising by weight:
(a) from about 50% to about 99.9% of a base oil being a volatile isoparaffin;
(b) from about 0.025% to about 15% of a silicone elastomer; and
(c) from about 1% to about 40% of a solubilizer for the silicone elastomer which is a non-volatile silicone having a viscosity from about 25 to about 100,000 mm²·s⁻¹.

The compositions of the present invention has improved stability such as reduced phase separation at lower temperature, and also has a balanced dry performance.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

### Composition

The composition of the present invention is a non-aqueous hair care composition comprising by weight:
(a) from about 50% to about 99.9% of a base oil being a volatile isoparaffin;
(b) from about 0.025% to about 15% of a silicone elastomer; and
(c) from about 1% to about 40% of a solubilizer for the silicone elastomer which is a non-volatile silicone having a viscosity from about 25 to about 100,000 mm²·s⁻¹.

It is believed that the use of isoparaffin base oil provides smoothness and shine, compared to compositions using other base oils such as volatile silicone base oils. It is also believed that the use of silicone elastomers provides less greasiness/stickiness and balanced hair volume to the isoparaffin base oil.

Furthermore, the inventors of the present invention have found that, in the isoparaffin base oil differently from volatile silicone base oil, phase separation may occur between the base oil and silicone elastomer, especially at a lower temperature and/or at a higher amount of the silicone elastomer. Silicone elastomer may be separated out from the isoparaffin base oil and form a visible particles and/or a different phase from the isoparaffin base oil phase, which is not desirable in view of product appearance and/or performance.

The inventors of the present invention have further found that, by the use of specific solubilizer for silicone elastomer in isoparaffin base oil, the composition of the present invention provides improved stability such as reduced phase separation, while providing the above balanced dry performance, compared to compositions using other solubilizers such as polydimethylsiloxane having a lower viscosity.

### Non Aqueous Composition

The composition of the present invention is a non-aqueous composition. Non-aqueous composition herein means that the composition is substantially free of water. In the present invention, “the composition being substantially free of water” means that: the composition is free of water; or, if the composition contains water, the level of water is very low. In the present invention, the level of water, if included, 1% or less, preferably 0.5% or less, more preferably 0.3% or less, still more preferably 0.1% or less, even more preferably 0% by weight of the composition.

### Turbidity

Preferably, the composition of the present invention, especially when the composition is liquid, has a homogeneous clear product appearance, i.e., homogeneous transparent product appearance.

Preferably, the liquid composition of the present invention has a turbidity of 2.0 NTU or less, 1.5 NTU or less, more preferably 1.0 NTU or less, still more preferably 0.7 NTU or less at 25° C. Further preferably, the composition of the present invention has such above turbidity at 25° C., even after its storage at 5° C. for at least 1 hour. The transmittances are measured by using HACH 2100N Turbidimeter.

In view of homogeneous clear product appearance, it is preferred to control the level of components which are substantially insoluble to the liquid composition of the present invention. By “substantially insoluble” compound, what is meant is that:

the compound is substantially insoluble in the compositions at the level used; and,
when containing the compounds at the level used, the compositions are either: (i) non-homogeneous by, for example, phase separation; or (ii) homogeneous but with a higher turbidity, i.e., turbidity of above 2.0 NTU (excluding 2.0 NTU), preferably above 1.5 NTU (excluding 1.5 NTU), more preferably above 1.0 NTU (excluding 1.0 NTU), still more preferably above 0.7 NTU (excluding 0.7 NTU) at 25° C.

Such substantially insoluble compounds further include fatty compounds including, for example: fatty alcohols having a melting point of 25° C. or more, such as cetyl alcohol and stearyl alcohol; fatty acids having a melting point of 25° C. or more, such as stearic acid; fatty alcohol derivatives and fatty acid derivatives such as esters and ethers thereof, which derivatives have a melting point of 25° C. or more; and mixtures thereof.

More preferably, the liquid compositions of the present invention are substantially free of such substantially insoluble compounds. In the present invention, the compositions being “substantially free” of substantially insoluble compounds means that: the composition is free of substantially insoluble compounds; or, if the composition contains substantially insoluble compounds, the level of such substantially insoluble compounds is very low. In the present invention, the level of such substantially insoluble compounds is, if included, 1.0% or less, preferably 0.5% or less, more preferably 0.3% or less, still more preferably 0.1% or less, even more preferably 0%.

### Substantially Free of Surfactants

Preferably, the composition of the present invention is substantially free of surfactants, in view of product clarity and/or reduced undesirable foaming of the product when the composition is liquid.

Such surfactants include: anionic surfactants; cationic surfactants; amphoteric surfactants; zwitterionic surfactants; nonionic surfactants having a HLB of 5 or more; and mixtures thereof. Such nonionic surfactants having a HLB of 5 or more includes, for example: ethers of fatty alcohols such as ethers of lauryl alcohol including Laureth-3 (having a HLB of about 8); esters of fatty alcohols such as esters of lauryl alcohols.

In the present invention, “the composition being substantially free of surfactants” means that: the composition is free of surfactants; or, if the composition contains surfactants, the level of such surfactants is very low. In the present invention, the total level of such surfactants is, if included, preferably 0.1% or less, more preferably 0.01% or less, still more preferably 0.001% or less, even more preferably 0.0005% by weight of the composition.

### Limited Amount of Volatile Silicone or Substantially Free of Volatile Silicone

In the composition of the present invention, if volatile silicone such as cyclomethicone is included, it is preferred to include it at a limited amount in view of balanced dry performance. For example, volatile silicone, if included, can be in the composition at a level preferably up to about 9%, more preferably up to about 5%, still more preferably up to about 3%, even more preferably up to about 1%. Further preferably, the composition of the present invention is free of volatile silicone.

### Base Oil

The composition of the present invention comprises a base oil. The base oil is included at a level by weight of the composition of, from about 50% to about 99.9%, from about 60% to about 99.8%, more preferably from about 65% to about 98%.

The base oil useful herein is a volatile isoparaffin, and liquid under 25° C.

### Volatile Isoparaffin

The volatile isoparaffin useful herein have one or several grade of isoparaffins and a viscosity of, preferably from about 0.5 mm²·s⁻¹ to about 50 mm²·s⁻¹, more preferably from about 0.8 mm²·s⁻¹ to about 40 mm²·s⁻¹, still more preferably from about 1 mm²·s⁻¹ to about 30 mm²·s⁻¹, even more preferably from about 1 mm²·s⁻¹ to about 20 mm²·s⁻¹, further more preferably from about 1 mm²·s⁻¹ to about 10 mm²·s⁻¹, at 37.8° C. When using two or more isoparaffin hydrocarbon solvents, it is preferred that the mixture of isoparaffin hydrocarbon solvents have the above viscosity.

Preferably, the volatile isoparaffin useful herein are those having from about 8 to about 20 carbon atoms, more preferably from about 8 to about 16 carbon atoms, still more preferably from about 10 to about 16 carbon atoms.

In the present invention, preferred volatile isoparaffin include, for example, trimer, tetramer, and pentamer of isobutene, and mixtures thereof. Commercially available isoparaffin hydrocarbons may have distributions of its polymerization degree, and may be mixtures of, for example, trimer, tetramer, and pentamer. What is meant by tetramer herein is that a commercially available isoparaffin hydrocarbons in which tetramer has the highest content, i.e., tetramer is included at a level of preferably 70% or more, more preferably 80% or more, still more preferably 85% or more.

### Silicone Elastomer

The composition of the present invention comprises a silicone elastomer. The silicone elastomer is included at a level by weight of the composition of, from about 0.025% to about 15%, preferably from about 0.1% to about 15%, more preferably from about 0.25% to about 10%, still more preferably from about 0.5% to about 5%, even more preferably from about 1% to about 5%.

The silicone elastomers useful herein are silicone chains which are crosslinked by silicone or hydrocarbon. Among them, preferred herein are those crosslinked by silicone, such as that having an INCI name Dimethicone/VinylDimethicone Crosspolymer which is available commercially for example as KSG-16 (ShinEtsu Silicone). Those crosslinked by hydrocarbon are, for example, that having an INCI name Dimethicone Crosspolymer which is available commercially for example as Dow Coming 9041 Silicone Elastomer Blend (Dow Corning Silicone).

Silicone elastomers are often supplied as a mixture with solvents such as cyclomethicone and a lower viscosity non-volatile silicone having a viscosity of from about 3 to about 10 cst. Such silicone elastomers supplied as a mixture with cyclomethicone include, for example, KSG-15 (ShinEtsu Silicone). Such silicone elastomers supplied as a mixture with the above lower viscosity non-volatile silicone include, for example, KSG-16 (ShinEtsu Silicone) and Dow Coming 9041 Silicone Elastomer Blend (Dow Coming Silicone). In the present invention, it is preferred to use a silicone elastomer supplied as a mixture with the lower viscosity non-volatile silicone. The solvent are used so that the weight ratio of the silicone elastomer to the solvent of from about 1:3 to about 1:10.

### Solubilizer for Silicone Elastomer

The composition of the present invention comprises a solubilizer for the silicone elastomer, in view of providing homogeneous clear product appearance while containing silicone elastomer.

The solubilizer is included at a level by weight of the composition of, preferably from about 1% to about 40%, more preferably from about 1% to about 30%, still more preferably from about 5% to about 20%. It is preferred to include the solubilizer at a level such that the weight ratio of the solubilizer to the silicone elastomer is from about 1:1 to about 30:1, more preferably from about 2:1 to about 20:1, still more preferably from about 3:1 to about 10:1,

The solubilizers useful herein are non-volatile silicone compounds, preferably non-volatile, non-amino modified silicone compounds, more preferably, polydimethysiloxane.

The solubilizer useful herein are those having a viscosity of from about 25 mm²·s⁻¹ to about 100,000 mm² s⁻¹, preferably from about 50 mm²·s⁻¹ to about 50,000 mm²·s⁻¹, more preferably from about 100 mm²·s⁻¹ to about 30,000 mm²·s⁻¹, at 25° C., still more preferably from about 7,000 mm²·s⁻¹ to about 20,000 mm²·s⁻¹.

### Esters

The compositions of the present invention may further contain an ester. The ester can be included in the composition by weight of, from about 0.1% to about 25%, preferably from about 0.5% to about 15%, more preferably from about 1% to about 10%, still more preferably from about 1% to about 5%, in view of the balance between conditioned feel and product stability, and/or in view of prevent foaming especially when the composition is liquid.

The esters useful herein are those having an HLB of 3 or less, and liquid at room temperature. The esters useful herein are, for example, those selected from the group consisting of methyl esters of C12-18 fatty acids and mixtures thereof. Such esters include, for example, methyl palmitate, methyl stearate, methyl oleate, methyl linoleate, and methyl laurate. Among them, methyl stearate is preferably used in the present invention. Such esters are believed to help the silicone conditioning agents dissolved into the base oil of the present invention.

### Silicone Conditioning Agent

The composition of the present invention may further contain a silicone conditioning agent. The silicone conditioning agent can be included at a level by weight of the composition of, from about 0.1%, preferably from about 0.2%, more preferably from about 0.5% in view of providing dry conditioning benefit such as reduced friction, and to about 15%, preferably to about 10%, more preferably to about 8%, still more preferably to about 5% in view of conditioning benefits and/or product clarity.

The silicone conditioning agents useful herein are non-volatile, and preferably liquid at 25° C. The silicone conditioning agents useful herein have an apparent viscosity at 25° C. of, from about 200 mm²·s⁻¹, preferably from about 200 to about 300,000 mm²·s⁻¹, more preferably from about 200 to about 50,000 mm²·s⁻¹, in view of providing conditioning benefits.

The silicone conditioning agents useful herein are hydrophobic and those having a HLB of about 8 or less, preferably 5 or less.

The silicone conditioning agents useful herein are, preferably, in non-emulsion form. Aminosilicone

The silicone conditioning agent is preferably an aminosilicone. Preferably, aminosilicones useful herein have an amine content of less than about 0.12 m mol/g, more preferably less than about 0.1 m mol/g, still more preferably less than about 0.08 m mol/g, even more preferably less than about 0.06 m mol/g, in view of friction reduction benefit. It has been surprisingly found that higher levels of nitrogen (amine functional groups) in the amino silicone tend to result in less friction reduction, and consequently less conditioning benefit from the aminosilicone.

Aminosilicone useful herein are those which conform to the general formula (I):

(R₁)ₐG₃₋ₐ-Si—(—OSiG₂)ₙ—(—OSiG_{b}(R₁)_{2-b})ₘ—O—SiG₃₋ₐ(R₁)ₐ

wherein G is hydrogen, phenyl, hydroxy, or C₁-C₈ alkyl, preferably methyl; a is an integer having a value from 1 to 3, preferably 1; b is 0, 1 or 2, preferably 1; n is a number from 1 to 2,000, preferably from 100 to 1,800, more preferably from 300 to 800, still more preferably 500-600; m is 0; R₁ is a monovalent radical conforming to the general formula C_{q}H_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion. L is preferably —N(CH₃)₂ or —NH₂, more preferably —NH₂.

One highly preferred amino silicones are those corresponding to formula (I) wherein m=0, a=1, q=3, G=methyl, n is preferably from about 1400 to about 1700, more preferably around 1600; and L is —N(CH₃)₂ or —NH₂, more preferably —NH₂. Another further highly preferred amino silicones are those corresponding to formula (I) wherein m=0, a=1, q=3, G=methyl, n is preferably from about 400 to about 800, more preferably about 500 to around 600; and L is —N(CH₃)₂ or —NH₂, more preferably —NH₂.

Such preferred aminosilicones can be called as terminal aminosilicones, as one or both ends of the silicone chain are terminated by nitrogen containing group. It is also surprisingly found that, such terminal aminosilicones provide improved friction reduction compared to graft aminosilicones.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as vitamin E, panthenol and its solubilizer such as lauryl alcohol, panthenyl ethyl ether, hydrolysed keratin, proteins, plant extracts, and nutrients; preservatives such as benzyl alcohol, methyl paraben, propyl paraben and imidazolidinyl urea; coloring agents, such as any of the FD&C or D&C dyes; perfumes; and antidandruff agents.

### Product Forms

The hair care compositions of the present invention can be hair conditioning and/or hair styling products, and the like.

The hair care compositions of the present invention are, preferably, in the form of leave-on products, and can be formulated in a wide variety of product forms, including but not limited to gels, sprays, oils, liquid and serum. Preferably, the compositions of the present invention are in a liquid form such as oil, liquid and serum.

### Method of Use

For a leave-on form, the hair care composition can be applied to wet or damp hair prior to drying of the hair. After such hair care compositions are applied to the hair, the hair is dried and styled in accordance with the preference of the user. In the alternative, it may be applied to already dry hair, and the hair is then combed or styled, and dried in accordance with the preference of the user.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

[Compositions]

| Components | Ex. 1 | Ex. 2 | Ex. 3 | Ex. 4 | Ex. 5 | Ex. 6 | Ex. 7 |
|---|---|---|---|---|---|---|---|
| Volatile isoparaffin-1 *1 | q.s. to 100% | q.s. to 100% | q.s. to 100% | q.s. to 100% | q.s. to 100% | q.s. to 100% | q.s. to 100% |
| Volatile isoparaffin-2 *2 | — | — | — | 20 | — | — | — |
| Cyclopentasiloxane | — | — | — | — | — | — | — |
| Silicone elastomer-1 *3 | 1 | 1 | 2.5 | 2.5 | 2.5 | 2.5 | 2.5 |
| Solvent of Silicone elastomer-1 *4 | 3 | 3 | 7.5 | 7.5 | 7.5 | 7.5 | 7.5 |
| Silicone elastomer-2 *5 | — | — | — | — | — | — | — |
| Solvent of Silicone elastomer-2 *6 | — | — | — | — | — | — | — |
| Solubilizer-1 *7 | 5 | 5 | 10 | 10 | — | — | — |
| Solubilizer-2 *8 | — | — | — | — | 10 | — | — |
| Solubilizer-3 *12 | — | — | — | — | — | 10 | — |
| Solubilizer-4 *13 | — | — | — | — | — | — | 10 |
| Solubilizer-5 *14 | — | — | — | — | — | — | — |
| Other Solubilizer-1 *9 | — | — | — | — | — | — | — |
| Panthenol | 0.01 | 0.01 | 0.01 | 0.01 | 0.01 | 0.01 | 0.01 |
| Lauryl alcohol | 1 | 1 | 1 | 1 | 1 | 1 | 1 |
| Aminosilicone *10 | 10 | 10 | 10 | 10 | 10 | 10 | 10 |
| Ester *11 | — | 10.0 | — | — | — | — | — |
| Perfume | 0.4 | 0.4 | 0.4 | 0.4 | 0.4 | 0.4 | 0.4 |
| Stability@low temp (5° C.) | — | — | ◯ | ◯ | ◯ | ◯ | ◯ |
| Smoothness | — | — | ⊚ | — | R-1 | R-1 | R-1 |
| Hair volume | — | — | ⊚ | — | H-1 | ⊚ | H-1 |
| Clumping | — | — | ⊚ | — | ⊚ | ⊚ | ⊚ |
| Hair Shine | — | — | ⊚ | — | LS-1 | LS-1 | LS-1 |

| Components | Ex. 8 | Ex. 9 | CEx. i | CEx. ii | CEx. iii |
|---|---|---|---|---|---|
| Volatile isoparaffin-1 *1 | q.s. to 100% | q.s. to 100% | — | q.s. to 100% | q.s. to 100% |
| Volatile isoparaffin-2 *2 | — | — | — | — | — |
| Cyclopentasiloxane | — | — | q.s. to 100% | — | — |
| Silicone elastomer-1 *3 | 2.5 | — | 2.5 | 2.5 | 2.5 |
| Solvent of Silicone elastomer-1 *4 | 7.5 | — | 7.5 | 7.5 | 7.5 |
| Silicone elastomer-2 *5 | — | 2.5 | — | — | — |
| Solvent of Silicone elastomer-2 *6 | — | 13 | — | — | — |
| Solubilizer-1 *7 | — | 10 | 10 | — | — |
| Solubilizer-2 *8 | — | — | — | — | — |
| Solubilizer-3 *12 | — | — | — | — | — |
| Solubilizer-4 *13 | — | — | — | — | — |
| Solubilizer-5 *14 | 10 | — | — | — | — |
| Other Solubilizer-1 *9 | — | — | — | — | 10 |
| Panthenol | 0.01 | 0.01 | 0.01 | 0.01 | 0.01 |
| Lauryl alcohol | 1 | 1 | 1 | 1 | 1 |
| Aminosilicone *10 | 10 | 10 | 10 | 10 | 10 |
| Ester *11 | — | | — | — | — |
| Perfume | 0.4 | 0.4 | 0.4 | 0.4 | 0.4 |
| Stability@low temp (5° C.) | ◯ | ◯ | ◯ | X | X |
| Smoothness | R-1 | — | R-1 | — | — |
| Hair volume | H-1 | — | H-2 | — | — |
| Clumping | ⊚ | — | ⊚ | — | — |
| Hair Shine | LS-1 | — | LS-2 | — | — |

Definitions of Components
*1 Volatile isoparaffin-1: Trimer of isobutene having a viscosity of 1-2 mm² · s⁻¹ at 37.8° C.
*2 Volatile isoparaffin-2: Tetramer of isobutene having a viscosity of 2-3 mm² · s⁻¹ at 37.8° C.
*3 Silicone elastomer-1: That crosslinked by silicones, and having an INCI name Dimethicone/Vinyl Dimethicone Crosspolymer, supplied as KSG-16 from Shin-Etsu Silicone
*4 Solvent of Silicone elastomer-1: 6 mm² · s⁻¹ Polydimethylsiloxane, supplied as a 75:25 mixture with Silicone elastomer-1 as KSG-16 from Shin-Etsu Silicone
*5 Silicone elastomer-2: That crosslinked by hydrocarbon, and having an INCI name Dimethicone Crosspolymer, supplied as Dow Corning 9041 Silicone Elastomer Blend from Dow Corning Silicone
*6 Solvent of Silicone elastomer-2: 6 mm² · s⁻¹ Polydimethylsiloxane supplied as 84:16 mixture with Silicone elastomer-2 as Dow Corning 9041 Silicone Elastomer Blend from Dow Corning Silicone
*7 Solubilizer-1: Polydimethylsiloxane having a viscosity of 10,000 mm² · s⁻¹
*8 Solubilizer-2: Polydimethylsiloxane having a viscosity of 5,000 mm² · s⁻¹
*9 Other Solubilizer-1: Polydimethylsiloxane having a viscosity of 20 mm² · s⁻¹
*10 Aminosilicone: Terminal aminosilicone which is available from GE having a viscosity of about 10,000 mm² · s⁻¹ at 25° C., and having following formula: (R₁)ₐG₃₋ₐ—Si—(—OSiG₂)ₙ—(—OSiG_{b}(R₁)_{2-b})ₘ—O—SiG₃₋ₐ(R₁)ₐ wherein G is methyl; a is an integer of 1; n is a number from 400 to about 600; m is an integer of 0; R₁ is a monovalent radical conforming to the general formula CqH_{2q}L, wherein q is an integer of 3 and L is —NH₂
*11 Ester: Methy stearate
*12 Solubilizer-3: Polydimethylsiloxane having a viscosity of 1,000 mm² · s⁻¹
*13 Solubilizer-4: Polydimethylsiloxane having a viscosity of 100 mm² · s⁻¹
*14 Solubilizer-5: Polydimethylsiloxane having a viscosity of 100,000 mm² · s⁻¹

### Method of Preparation

The hair oil compositions of “Ex. 1” through “Ex. 9” and “CEx. i” through “CEx. iii” as shown above can be prepared by any conventional method well known in the art. They are suitably made as follows:

Silicone elastomer, its solubilizer, and if included, a silicone conditioning agent and an ester are added to a base oil with agitation at room temperature until homogenized. If included, other components such as perfumes are added to the mixture with agitation.

### Properties and Conditioning Benefits

Stability and Dry performance (Performance on dry hair) are measured for some of the example formulations by the following methods. The results are shown above.

### Stability

Stability is evaluated by the composition appearance at 25° C., after its storage at 5° C. for 3 months.

◯: No phase separation was observed

×: Phase separation was observed

### Dry Performance

The composition is used for this dry performance before its exposure of 5° C. storage for the above stability test. Dry Performance is evaluated by sensory test using a hair sample by more than 6 panelists. The hair sample is treated by 0.25 ml/10 g hair of the composition, and kept for 6 hours for drying, then tested. Test results from the panelist are gathered and its average is obtained, and categorized as shown in below table. Dry conditioning is mainly evaluated by “Smoothness”, “Hair volume” especially by managing hair volume naturally, and also “Hair Shine” especially by having a good shine. Less stickiness/greasiness is evaluated by “Hair clumping”, and also “Hair shine” especially whether or not they have greasy shine or not.

| | |
|---|---|
| Smoothness | ⊚: Smooth as Ex. 3 when combing through by finger |
| | R-1: Slightly rougher when combing through by finger, |
| | compared to Ex. 3 |
| | R-2: Too rough when combing through by finger, |
| | compared to Ex. 3 |
| Hair Volume | H-2: Too high hair volume compared to Ex. 3 |
| | H-1: Slightly high hair volume compared to Ex. 3 |
| | ⊚: Manage Hair volume naturally as Ex. 3 |
| | L-1: Slightly low hair volume compared to Ex. 3 |
| | L-2: Too low hair volume compared to Ex. 3 |
| Hair Clumping | ⊚: No Hair Clumping each other of hair fibers as Ex. 3 |
| | C-1: Slightly Clumping Hair each other of hair fibers, |
| | compared to Ex. 3 |
| | C-2: Hair fibers are clumped each other very much, |
| | compared to Ex. 3 |
| Hair Shine | GS-2: Too much greasy shine compared to Ex. 3 |
| | GS-1: Slightly greasy shine compared to Ex. 3 |
| | ⊚: Hair has good shine as Ex. 3, which is not greasy |
| | shine from oil on the Hair surface |
| | LS-1: Hair has slightly reduced shine compared to Ex. 3 |
| | LS-2: Hair has significantly reduced shine compared |
| | to Ex. 3 |

Examples 1 through 9 are compositions of the present invention which are particularly useful for leave-on use. The embodiments disclosed and represented by the previous “Ex. 1” through “Ex. 9” have many advantages. For example, they provide a balanced dry performance. They also provide improved stability, especially reduced phase separation of silicone elastomer at lower temperature. They also have a clear product appearance.

Such advantages can be understood by the comparison between the examples of the present invention and comparative examples “CEx. i” through “CEx. iii”. For example, the comparative example “CEx.i”, which is almost identical to “Ex. 3” of the present invention except for containing a different base oil, shows too high hair volume, and significantly reduced shine. The comparative example “CEx.ii” is almost identical to “Ex. 3” of the present invention except for not containing any solubilizer. The comparative example “CEx.iii” is almost identical to “Ex. 3” of the present invention except for containing a different solubilizer. However, both of the compositions of the comparative examples “CEx. ii” and “CEx.iii” show phase separation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A non-aqueous hair care composition comprising by weight:
(a) from about 50% to about 99.9% of a base oil being a volatile isoparaffin;
(b) from about 0.025% to about 15% of a silicone elastomer; and
(c) from about 1% to about 40% of a solubilizer for the silicone elastomer which is a non-volatile silicone having a viscosity from about 25 to about 100,000 mm²·s⁻¹.

2. The composition of claim 1 wherein the non-volatile silicones having a viscosity from about 50 to about 50,000 mm²·s⁻¹.

3. The composition of claim 1 wherein the non-volatile silicones having a viscosity from about 100 to about 30,000 mm²·s⁻¹.

4. The composition of claim 1 wherein the non-volatile silicones having a viscosity from about 7,000 to about 20,000 mm²·s⁻¹.

5. The composition of claim 1 wherein the non-volatile silicones is a non-volatile non-amino modified silicone.

6. The composition of claim 1 wherein the non-volatile silicones is polydimethylsiloxane.

7. The composition of claim 1 wherein the weight ratio of the solubilizer to the silicone elastomer is from about 1:1 to about 30:1.

8. The composition of claim 1 wherein the weight ratio of the solubilizer to the silicone elastomer is from about 2:1 to about 20:1.

9. The composition of claim 1 wherein the weight ratio of the solubilizer to the silicone elastomer is from about 3:1 to about 10:1.

10. The composition of claim 1 wherein the composition is substantially free of surfactants.

11. The composition of claim 1 wherein the composition is for leave-on use.
